(19) United States
(12) Patent Application Publication
(10) Pub. No.: US 2017/0315114 A1
(43) Pub. Date: Nov. 2, 2017

(54) BIMOLECULAR AUTOINHIBITED BIOSENSOR

(71) Applicant: THE UNIVERSITY OF QUEENSLAND, St. Lucia, Queensland (AU)

(72) Inventors: Viktor STEIN, St Lucia, Queensland (AU); Kirill ALEXANDROV, St Lucia, Queensland (AU)

(21) Appl. No.: 15/521,971

(22) PCT Filed: Oct. 27, 2015
(86) PCT No.: PCT/AU2015/050669

§ 371 Date: Apr. 26, 2017

(30) Foreign Application Priority Data
Oct. 27, 2014 (AU) 2014904282

(51) Int. Cl. G01N 33/542

(52) U.S. Cl. CPC G01N 33/542

(57) ABSTRACT

A biosensor comprises first and second molecular components and is capable of displaying non-protease enzyme activity in response to a binding event mediated by first and second binding partners of the biosensor. The first and second binding partners may bind each other directly or may both bind a target molecule. At least the first molecular component comprises an inhibited non-protease enzyme, whereby the binding event switches the enzyme from a catalytically inactive state to an active state. The second molecular component may comprise a protease that cleaves the first molecular component to release inhibition of the non-protease enzyme of first molecular component. Alternatively, the second molecular component may comprise a trap molecule that binds a bait molecule of the first molecular component to release inhibition of the non-protease enzyme of first molecular component.

### TECHNICAL FIELD

THIS INVENTION relates to biosensors. More particularly, this invention relates to a biosensor comprising a non-protease enzyme activity that is suitable for selective detection of one or more target molecules. The biosensor may be used to detect molecules in biological, clinical, environmental and industrial samples. The biosensor may also relate to the field of synthetic biology such as for constructing artificial cellular or extracellular signalling networks.

### BACKGROUND

Detection of target molecules or analytes is a key to understanding and controlling complex biological processes such as organismal growth, metabolism, differentiation, cell- and life cycle progression, disease or death. Key requirements of analyte detection are specificity and sensitivity, particularly when the target molecule or analyte is in a limiting amount or concentration in a biological sample.

Typically, specificity is provided by monoclonal antibodies which specifically bind the analyte. Sensitivity is typically provided by a label bound to the specific antibody, or to a secondary antibody which assists detection of relatively low levels of analyte. This type of diagnostic approach has become well known and widely used in the enzyme-linked immunosorbent sandwich assay (ELISA) format. In some cases, enzyme amplification can even further improve sensitivity such as by using a product of a proenzyme cleavage reaction catalyzing the same reaction. Some examples of such “autocatalytic” enzymes are trypsinogen, pepsinogen, or the blood coagulation factor XII. However, in relation to specificity antibodies are relatively expensive and can be difficult to produce with sufficient specificity for some analytes. Polyclonal antibodies also suffer from the same shortcomings and are even more difficult to produce and purify on a large scale.

Current methods to detect specific target molecules and analytes for either prognostic or diagnostic purposes suffer from a number of limitations which significantly restrict their widespread application in clinical, peri-operative and point-of-care settings. Most importantly, the vast majority of diagnostic assays require a significant level of technical expertise and a panel of expensive and specific reagents (most notably monoclonal antibodies) along with elaborate biomedical infrastructures which are rarely available outside specialized laboratory environments. For instance, ELISAs—the gold standard for detecting specific analytes in complex biological samples—rely on the selective capture of a target analyte on a solid surface which in turn is detected with a second affinity reagent that is specific for the target analyte. ELISAs also feature extensive incubation and washing steps which are generally time consuming and difficult to standardize as the number of successive steps frequently introduces significant variation across different procedures, operators and laboratories making quantitative comparisons difficult.

### SUMMARY

The present invention addresses a need to develop a quantitative, relatively inexpensive and easily produced molecular biosensor that readily detects the presence or the activity of target molecules rapidly and sensitively. It is also an objective to produce a molecular biosensor that has broad applicability in cellular engineering, molecular diagnostics, drug screening, biomarker detection and other applications that require detection of binding events or enzymatic activities.

A particular aspect of the invention relates to the use of an enzyme other than a protease for generating a detectable signal in response to, or upon, a binding event. An advantage of this biosensor is that the catalytic rate of the non-protease enzyme can be much higher than that of a protease, hence the responsiveness and sensitivity of the biosensor is superior as is the ability to provide any of a variety of different outputs including chromogenic, electrochemical and other detectable signals.

In one broad form the invention relates to a biosensor that comprises a first molecular component and a second molecular component, wherein the first molecular component comprises a first binding partner, an amino acid sequence of an enzyme that is not a protease, and an inhibitor of the enzyme and wherein the second molecular component is capable of facilitating at least partial release of inhibition of the enzyme of the first molecular component by the inhibitor to thereby switch the enzyme of the first molecular component from a catalytically inactive to a catalytically active state.

In one aspect, the invention relates to a biosensor that comprises a first molecular component and a second molecular component, wherein the first molecular component comprises: a first binding partner, an amino acid sequence of an enzyme that is not a protease; and an inhibitor of the enzyme; and the second molecular component comprises: a second binding partner and an amino acid sequence capable of facilitating at least partial release of inhibition of the enzyme of the first molecular component by the inhibitor to thereby switch the enzyme of the first molecular component from a catalytically inactive to a catalytically active state.

Suitably, the enzyme of the first molecular component is any enzyme which is at least partially inhibitable and switchable from a catalytically inactive to a catalytically active state. Suitably, in a catalytically active state the enzyme is capable of acting upon, or reacting with, a substrate to produce a detectable signal. The detectable signal may be chromogenic, fluorescent, light (e.g. bioluminescent), electrical, radioactive and other detectable signals.

In one general embodiment, the amino acid sequence of the second molecular component is of a protease. According to this general embodiment, the protease of the second molecular component is capable of proteolytically cleaving a protease cleavage site in the first molecular component. Suitably, this cleavage event is capable of facilitating at least partial release of inhibition of the enzyme of the first molecular component by the inhibitor to thereby switch the enzyme of the first molecular component from a catalytically inactive to a catalytically active state.

The protease may be a constitutively active protease or an at least partly inhibited protease. Suitably, the second molecular component comprises an inhibitor, preferably an autoinhibitor, that at least partly inhibits the protease. In some embodiments, the inhibitor is a peptide. A preferred inhibitor peptide is an autoinhibitory peptide.

In one embodiment, the protease of the second molecular component is an endopeptidase. Preferably, the endopeptidase is a cysteine protease.

In an embodiment, the protease is derivable or obtainable from a virus.

In certain embodiments the virus is a Potyvirus such as, tobacco vein mottling virus (TVMV), tobacco etch virus (TEV) or sugarcane mosaic virus (SMV) or a Flavivirus such as Hepatitis C Virus (HCV) or a small ubiquitin-like modifier (SUMO) protease.

Preferably, the protease is an NIa protease.

In some particular embodiments, the autoinhibitor peptide is a peptide autoinhibitor of an NIa protease of a Potyvirus.

In other particular embodiments, the protease is of a SUMO protease.

In another general embodiment, the amino acid sequence of the second molecular component is not of a protease. In one embodiment, the amino acid sequence of the second molecular component is of a trap protein capable of binding a bait protein of the first molecular component. Suitably, binding of the bait protein by the trap protein facilitates release of inhibition of the enzyme of the first molecular component to thereby switch the enzyme of the first molecular component from a catalytically inactive to a catalytically active state.

In some embodiments, the first binding partner and the second binding partner may be capable of binding, coupling, interacting or forming a complex with a target molecule to thereby co-localize the first molecular component and the second molecular component to facilitate at least partial release of inhibition of the enzyme of the first molecular component.

In other embodiments, the first binding partner and the second binding partner may be capable of directly binding, coupling, interacting or forming a complex to thereby co-localize the first molecular component and the second molecular component to facilitate at least partial release of inhibition of the enzyme of the first molecular component.

Suitably, the first binding partner and the second binding partner are different molecules (e.g. proteins, nucleic acids, sugars, lipids or combinations of these although without limitation thereto) or are different portions, parts, segments, moieties, domains, regions, sub-sequences or fragments of the same molecule.

In a preferred form, the first molecular component and the second molecular component are separate, recombinant fusion proteins. Amino acid sequences of particular embodiments of the first molecular component, second molecular component and constituent subcomponents, proteases, protease inhibitors, cross-binders and other portions thereof are set forth in SEQ ID NOS:1 and 2. Also provided are fragments, derivatives and variants of the amino acid sequences set forth in SEQ ID NOS:1 and 2.

Another aspect of the invention provides a composition or kit comprising the biosensor of the aforementioned aspect and a substrate.

The composition or kit may comprise one or a plurality of different biosensors disclosed herein capable of detecting one or a plurality of different target molecules.

A further aspect of the invention provides a method of detecting a binding interaction between the first and second molecular components of the biosensor of the aforementioned aspect, said method including the step of contacting the composition of the aforementioned aspect with a sample to thereby determine the presence or absence of the target molecule in the sample.

Another further aspect of the invention provides a method of detecting a target molecule, said method including the step of contacting the biosensor of the aforementioned aspect with a sample to thereby determine the presence or absence of the target molecule in the sample.

A still further aspect of the invention provides a method of diagnosis of a disease or condition in an organism, said method including the step of contacting the of the biosensor of the aforementioned aspect with a biological sample obtained from the organism to thereby determine the presence or absence of a target molecule in the biological sample, determination of the presence or absence of the target molecule facilitating diagnosis of the disease or condition.

The organism may include plants and animals inclusive of fish, avians and mammals such as humans.

A still yet further aspect of the invention provides a detection device that comprises a cell or chamber that comprises the biosensor of the first aspect.

Suitably, a sample may be introduced into the cell or chamber to thereby facilitate detection of a target molecule.

In certain embodiments, the detection device is capable of providing an electrochemical, acoustic and/or optical signal that indicates the presence of the target molecule.

The detection device may further provide a disease diagnosis from a diagnostic target result by comprising:
a processor; and
a memory coupled to the processor, the memory including computer readable program code components that, when executed by the processor, perform a set of functions including:
analysing a diagnostic test result and providing a diagnosis of the disease or condition.

The detection device may further provide for communicating a diagnostic test result by comprising:
a processor; and
a memory coupled to the processor, the memory including computer readable program code components that, when executed by the processor, perform a set of functions including:
transmitting a diagnostic result to a receiving device; and
optionally receiving a diagnosis of the disease or condition from the or another receiving device.

A related aspect of the invention provides an isolated nucleic acid encoding the first molecular component and/or the second molecular component of the biosensor of the aforementioned aspect.

Another related aspect of the invention provides a genetic construct comprising the isolated nucleic acid of the aforementioned aspect.

A further related aspect of the invention provides a host cell comprising the genetic construct of the aforementioned aspect.

It will be appreciated that the indefinite articles “a” and “an” are not to be read as singular indefinite articles or as otherwise excluding more than one or more than a single subject to which the indefinite article refers. For example, “a” molecule includes one molecule, one or more molecules or a plurality of molecules.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

FIG. 1: Overview of general embodiments of bimolecular biosensors. In all embodiments the first molecular component comprises a non-protease enzyme in an initially inhibited state. A: Bimolecular biosensor where the second molecular component comprises a constitutively active protease and the first molecular component comprises a cleavage site for the protease, arranged so that cleavage activates the non-protease enzyme. B: Bimolecular biosensor where the second molecular component comprises a partly autoinhibited protease and the first molecular component comprises a cleavage site for the protease, arranged so that cleavage activates the non-protease enzyme. (C) Bimolecular biosensor where the second molecular component comprises a trap protein and the first molecular component comprises a bait molecule, whereby binding of the bait molecule of by the trap molecule activates the non-protease enzyme.

FIG. 2: An embodiment of a bimolecular biosensor wherein the first and second binding partners are respectively, rapamycin-binding proteins FRB and FKBP12. The non-protease enzyme of the first molecular component is β lactamase and the inhibitor is β lactamase inhibitory peptide (BLIP). The protease of the second molecular component is a partly autoinhibited TVMV protease. The first molecular component comprises a cleavage site for TVMV.

FIG. 3: Rapamycin-induced biosensor activation via TVMV cleavage of a TVMV cleavage site in the first molecular component. Concentration of first molecular component (BLA-BLIP-FRB) is 5 nM (upper panels) or 1 nM (lower panels). Concentration of second molecular component (FKBP12-TVMV) is 10 nM. Substrate is nitrocefin at 25 μM. Background Subtraction: 1 nM BLA-BLIP-FRB; 0 nM TVMV-FRB. Biosensor activation was measured in the absence or presence of increasing concentrations of rapamycin (0.025 nM-50 nM).

### DETAILED DESCRIPTION

The present invention provides a biosensor comprising first and second molecular components which is capable of displaying enzymatic activity in response to a binding event. Suitably the first and second molecular components respectively comprise first and second binding partners. The first and second binding partners may bind each other directly or may both bind a target molecule. This binding event brings the first and second molecular components into the proximity of each other to switch the enzyme of the first molecular component from an “inactive” state to an “active” state. Suitably, the first molecular component comprises an inhibited enzyme that is not a protease. The second molecular component comprises an amino acid sequence capable of facilitating at least partial release of inhibition of the enzyme of the first molecular component. In one general embodiment, the amino acid sequence of the second molecular component is of a protease which may be constitutively active or partly autoinhibited, whereby the binding event mediated by the first and second binding partners switches the enzyme of the first molecular component from an inhibited “inactive” state to an “active” state. In another general embodiment, the amino acid sequence of the second molecular component is of a trap protein that can bind or interact with a bait molecule of the first molecular component, whereby binding of the bait molecule by the trap protein releases the enzyme inhibitor to switch the enzyme of the first molecular component from an inhibited “inactive” state to an “active” state. The enzymatically activate biosensor of either embodiment may act upon a substrate to facilitate generation of a detectable signal.

The biosensor disclosed herein may have particular efficacy in molecular diagnostics or analytics wherein the target molecule is an analyte or other molecule of diagnostic value or other importance. However, another application of the biosensor disclosed herein may be in synthetic biology applications for constructing multi-component artificial cellular signalling networks regulating cellular processes, the detection of protein:protein, protein:small molecule interactions, antibody-mediated detection of antigens and biomarkers as well as the construction of in vivo screening and selection systems comparable to two hybrid systems.

For the purposes of this invention, by “isolated” is meant material (such as a molecule) that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated proteins and nucleic acids may be in native, chemical synthetic or recombinant form.

By “protein” is meant an amino acid polymer. The amino acids may be natural or non-natural amino acids, D- or L-amino acids as are well understood in the art.

A “peptide” is a protein having less than fifty (50) amino acids.

A “polypeptide” is a protein having fifty (50) or more amino acids.

An aspect of the invention provides a biosensor that comprises a first molecular component and a second molecular component, wherein the first molecular component comprises: a first binding partner, an amino acid sequence of an enzyme that is not a protease; and an inhibitor of the enzyme; and the second molecular component comprises: a second binding partner and an amino acid sequence capable of facilitating at least partial release of inhibition of the enzyme of the first molecular component by the inhibitor to thereby switch the enzyme of the first molecular component from a catalytically inactive to a catalytically active state.

While the terms “first” and “second” are used in the context of respective, separate or discrete molecular components and/or first and second binding partners of the biosensor, it will be appreciated that these do not relate to any particular non-arbitrary ordering or designation that cannot be reversed. Accordingly, the structure and functional properties of the first molecular component and the second molecular component disclosed herein could be those of a second molecular component and a first molecular component, respectively. Similarly, the structure and functional properties of the first binding partner and the second binding partner disclosed herein could be those of a second binding partner and a first binding partner, respectively. It will also be appreciated that the biosensor may further comprise one or more other, non-stated molecular components.

In this context, a “molecular component” is a discrete molecule that forms a separate part, portion or component of the biosensor. In typical embodiments, each molecular component is, or comprises, a single, contiguous amino acid sequence (i.e a fusion protein). While it will be apparent that in many embodiments the first and second molecular components may non-covalently bind, couple, interact or associate in the context of a “binding event” mediated by the first and second binding partners, they remain discrete molecules that form the biosensor.

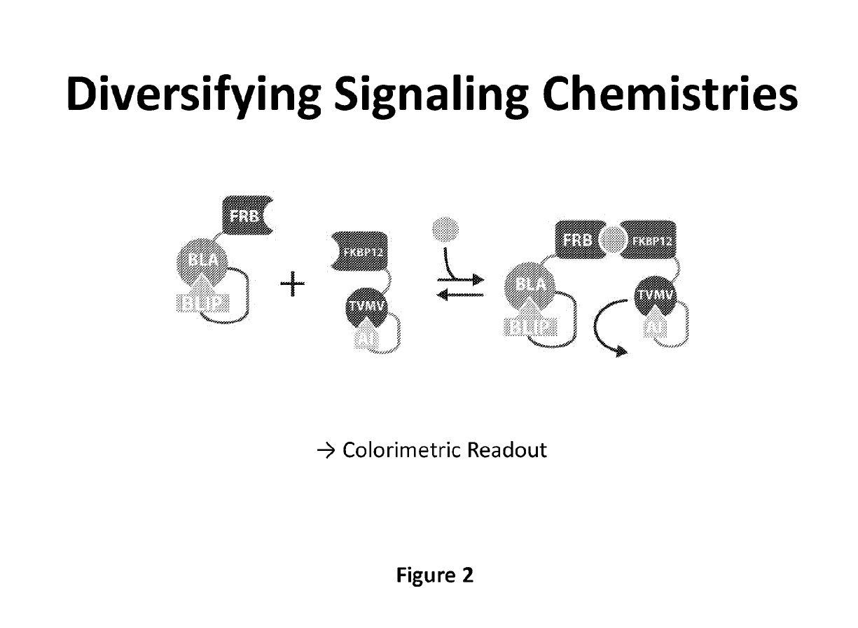

Suitably, the first molecular component comprises an amino acids sequence of an enzyme that is not a protease. Suitably, the enzyme is capable of being reversibly inhibited, such as by an autoinhibitor. By this is meant that the catalytic activity of the enzyme may be at least partially inhibited, which partial inhibition may be released or removed to thereby switch the enzyme of the first molecular component from an at least partly inhibited or catalytically “inactive” state to an at least partly catalytically “active” state. In this context, it will be appreciated that “active” and “inactive” respectively refer to, and include, completely or at least partially catalytically active and inactive states of the enzyme. Suitably, the catalytically active enzyme is capable of reacting with or acting upon a substrate molecule to thereby elicit a detectable signal. Non-limiting examples of enzymes include β-lactamase, β-galactosidase, glucose oxidase, lysozyme, malate dehydrogenase, peroxidases (e.g HRP), phosphatases (e.g alkaline phosphatase), luciferase, transferases, ATPases, nucleases (e.g. ribonucleases), kinases, synthases, oxidoreductases and dehydrogenases such as glucose dehydrogenase, flavin adenine dinucleotide-dependent glucose dehydrogenase (FADGDH) and pyranose dehydrongease (PDH), although without limitation thereto. Non-limiting examples of substrates include those that enable the generation of chromogenic, fluorescent, light (e.g. bioluminescent), electrical, radioactive and other detectable signals. One particular embodiment provided by the present invention is a first molecular component comprising an amino acid sequence of b-lactamase and a β-lactamase inhibitor such as β-lactamase inhibitory peptide (“BLIP”), as for example shown in FIG. 2. A non-limiting amino acid sequence of a first molecular component comprising β-lactamase and BLIP amino acid sequences is set forth in SEQ ID NO:1. Other particular embodiments may relate to an enzyme capable of reacting with a substrate molecule to thereby produce one or more electrons. Preferably, the enzyme is an oxidoreductase such as GDH, LDH or DHFR. Another particular embodiment may relate to an enzyme capable of reacting with a substrate molecule to thereby produce one or more photons. Preferably, the enzyme is a luciferase such as NanoLuc (e.g see Hall et al., 2012, ACS Chem Biol. 7 1848-57) which can be converted into a protease-cleavable zymogen using luciferase-based inhibitors such as coelenteramide by means of chemo-enzymatic coupling of benzyl-guanine-coelenteramide derivatives to protease-cleavable fusion proteins of NanoLuc and SNAP-tag (e.g. Schena et al., 2015, Nature Communications 6 7830). Examples of artificial zymogens activatable by proteases may include ribonuclease (Plainkum et al., 2003, Nat Struct Biol 10 115-119), adenosine diphosphate (ADP)-ribosyltransferase (Jucovic et al., 2008, Protein Eng Des Sel 21 631-638) and barnase (Mitrea et al., 2010, Proc Natl Acad Sci USA 107 2824-2829).

In one general embodiment, the second molecular component of the biosensor comprises an amino acid sequence of a protease.

A “protease” is any protein which displays, or is capable of displaying, an ability to hydrolyse or otherwise cleave a peptide bond. Like terms include “proteinase” and “peptidase”. Proteases include serine proteases, cysteine proteases, metalloproteases, threonine proteases, aspartate proteases, glutamic acid proteases, acid proteases, neutral proteases, alkaline proteases, exoproteases, aminopeptidases and endopeptidases although without limitation thereto. Proteases may be purified or synthetic (e.g. recombinant synthetic) forms of naturally-occurring proteases or may be engineered or modified proteases which comprise one or more fragments or domains of naturally-occurring proteases which, optionally, have been further modified to possess one or more desired characteristics, activities or properties.

According to this general embodiment, the first molecular component suitably comprises an amino acid sequence cleavable by the protease of the second molecular component. Suitably, cleavage of the amino acid sequence is capable of facilitating at least partial release of inhibition of the enzyme of the first molecular component by the inhibitor to thereby switch the enzyme of the first molecular component from a catalytically inactive to a catalytically active state.

The protease may be a constitutively active protease or an at least partly inhibited protease. Suitably, the second molecular component comprises an autoinhibitor that at least partly inhibits the protease. Employing a partially autoinhibited protease in the second molecular component can help reduce non-specific background activation “noise” in the absence of the target molecule In some embodiments, the inhibitor is a peptide. Alternatively, a constitutively active protease (i.e in the absence of an inhibitor) can provide greater signal amplification. A preferred inhibitor peptide is an autoinhibitory peptide.

The protease amino acid sequence of the second molecular component may be an entire amino acid sequence of a protease or may be an amino acid sequence of a proteolytically-active fragment or sub-sequence of a protease.

In one preferred embodiment, the protease is an endopeptidase.

Preferably, the endopeptidase is a cysteine protease or serine protease. A particular example of a cysteine protease is NIa protease of Potyviruses. A particular example of a serine protease is an NS3 protease of a Flavivirus such as HC.

In another preferred embodiment, the protease is a naturally-occurring protease.

A preferred class of proteases are derived from, or encoded by, a viral genome.

Typically, such proteases are dependent on expression and proteolytic processing of a polyprotein and/or other events required as part of the life cycle of viruses such as Picornavirales, Nidovirales, Herpesvirales, Retroviruses and Adenoviruses, although without limitation thereto. Particular examples of proteases include: Potyviridae proteases such as the NIa protease of tobacco etch virus (TEV), tobacco vein mottling virus (TVMV), sugarcane mosaic virus (SMV) etc; Flaviviridae proteases such as the NS3 protease of hepatitis C virus (HCV); Picornaviridae proteases such as the 3C protease of EV71, Norovirus etc, the 2A protease of human rhinovirus, coxsackievirus B4 etc and the leader protease of foot and mouth disease virus (FMDV) etc; Coronaviridae proteases such as the 3C-like protease of SARS-CoV, IBV-CoV and Herpesvirus proteases such as HSV-1, HSV-2, HCMV and MCMV proteases etc, SUMO family proteases although without limitation thereto.

Preferably, the viral genome is of a plant virus.

More preferably, the plant virus is a Potyvirus.

In a particularly preferred embodiment, the protease is an auto-inhibited Potyvirus protease such as the NIa protease of TEV, TVMV or SMV.

In an alternative embodiment the protease is an autoinhibited NS3 protease of HCV.

The native function of NIa proteases from Potyviridae is to process the viral polyprotein proteome. Auto-inhibition is mediated by peptides that bind the active site of NIa proteases and inhibit their activity. Such inhibitors are typically derived from Site F which separates the NIb RNA polymerase from the viral coat protein, and is considered the most efficient substrate for NIa proteases.

In other embodiments, the protease is a SUMO protease. SUMO proteases may have a higher catalytic rate which is particularly advantageous for signal amplification. By way of example, reference is made to Frey & Görlich, 2014, J Chromatogr A 1337 95-105 and GenBank Accession Number AF151697.2 which provides an amino acid sequence for human sentrin-specific protease 2 (SENP2) and the encoding nucleotide sequence.

The protease inhibitor may be any molecule which at least partly, or substantially or totally suppresses or inhibits the protease activity of the amino acid sequence of the protease. The inhibitor may be a non-specific inhibitor by virtue of having inhibitory activity towards a plurality of different proteases or types of protease, or may be a specific inhibitor by virtue of substantially inhibiting only a single protease.

The protease inhibitor may be a protein (inclusive of peptides) or a non-protein organic molecule such as a small organic molecule, a lipid, a carbohydrate or a nucleic acid, although without limitation thereto.

Non-limiting examples of protease inhibitors that are proteins include viral autoinhibitory peptides, aprotinin, leupeptin, metallocarboxypeptidase A inhibitor, α2 macroglobulin, pepstatin and serpins such as alpha 1-antitrypsin, C1-inhibitor, antithrombin, alpha 1-antichymotrypsin, plasminogen activator inhibitor-1 and neuroserpin, although without limitation thereto. Inhibitors can comprise specific antibody or antibody fragments displaying inhibitory activity, protein domains or peptides displaying specific binding to the protease and exerting competitive, steric or allosteric inhibition, DNA, PNA or RNA aptamers capable of binding to the protease and exerting competitive, steric or allosteric inhibition.

Non-limiting examples of protease inhibitors that are organic molecules include phenylmethanesulfonyl fluoride, tosyl lysine chloromethylketone, tosyl phenylalanyl chloromethyl ketone, bestatin and nitrophenol-p-guanidino benzoate, phosphoramidite and protease inhibitors developed as antiviral agents, such as for treatment of HIV or hepatitis C infection. Non-limiting examples of antiviral protease inhibitors include ritonivir, saquinavir, indinavir, nelfinavir, tipranavir, amprenavir and daurnavir, although without limitation thereto.

Suitably, the protease inhibitor is a reversible protease inhibitor.

The inhibitor may be an active site inhibitor or an allosteric inhibitor of the protease.

Preferably, the protease inhibitor is an autoinhibitory peptide. Suitably, the autoinhibitory peptide comprises an amino acid sequence which binds the active site of a protease without being cleaved by the protease. Preferably, the autoinhibitory peptide competitively at least partly inhibits binding and cleavage of one or more protease substrates by the protease. In one embodiment, the autoinhibitory peptide is a specific inhibitor of an endopeptidase such as a cysteine protease. In a preferred embodiment, the autoinhibitory peptide is a specific inhibitor of a protease, preferably a cysteine protease, encoded by a viral genome.

More preferably, the autoinhibitory peptide is an inhibitor of a protease encoded by a Potyviral genome.

One particular embodiment of an autoinhibitory peptide is a specific inhibitor of a Potyvirus NIa protease, preferably encoded by a TEV, TVMV or SMV genome. Peptides that bind the active site of NIa proteases and inhibit their activity are generally derived from Site F which refers to a peptide sequence which separates the NIb RNA polymerase from the viral coat protein, and is considered the most efficient substrate for NIa proteases.

The autoinhibitory peptide may comprise an amino acid sequence that corresponds to at least a fragment of a substrate of the protease, but not an amino acid sequence of a protease cleavage site. In this regard, the autoinhibitory peptide may comprise an amino acid sequence that corresponds to that of a cleavage product or comprise an amino acid sequence of a protease cleavage site modified or engineered to resist cleavage by the protease.

In some embodiments, to improve binding of the autoinhibitory peptide to the protease, and thus achieve improved autoinhibition, one or more amino acid sequence mutations may be introduced into the amino acid sequence of the protease and/or the autoinhibitor. As will be described in more detail hereinafter in the Examples in embodiments relating to NIa protease of TVMV, modification of residues in the ‘RETVRFQSDT’ (SEQ ID NO: 3) of the site F autoinhibitory peptide may improve auto-inhibition while minimizing or eliminating cleavage by TVMV protease.

Binding of the autoinhibitory peptide can also be improved by improving the linker region connecting the autoinhibitory (AI) domain to the NIa protease, such as by truncating the C-terminus of TVMV and increasing the effective concentration of the AI domain near the active site.

In other embodiments, autoinhibition can be improved by introducing beneficial steric constraints either through specific dimerization modules located at the N- and C-terminus of the protease biosensor or by circular permutation. Circularly permutated protease biosensors may feature two linker sites which can incorporate recognition sites for two different target proteases.

Persons skilled in the art will appreciate that the modifications described above in relation to NIa proteases and autoinhibitory peptides may be applied in principle to other proteases and/or autoinhibitory peptides suitable for use in biosensors.

For example, in a manner analogous to NIa proteases, artificially autoinhibited signal transducers based on HCV can be created by joining the peptide-based active site binder DELILCPLDL (SEQ ID NO:4) to its C-terminus via a linker comprising a TVMV cleavage site.

In another preferred embodiment the protease is a SUMO family protease and the inhibitor such as SUMO and SUMO like domains linked via a peptide linker indigestible by SUMO protease.

Suitably, the protease amino acid sequence and the first binding partner amino acid sequence are contiguous, or optionally, connected by a linker amino acid or amino acid sequence. The first binding partner amino acid sequence may be contiguous or linked to the N- or C-terminal amino acid of the protease amino acid sequence.

In embodiments where the inhibitor of protease activity comprises an amino acid sequence (i.e. is a protein or peptide), this is preferably fused or connected to the protease amino acid sequence by a linker amino acid sequence. In some embodiments, the linker amino acid sequence is, or comprises a bait amino acid sequence, as will be described in more detail hereinafter.

Other particular embodiments of the second molecular component may include circularly permutated protease constructs and split protease constructs such as described in WO2014/040129, although without limitation thereto.

In embodiments where the inhibitor of protease activity does not comprise an amino acid sequence (e.g. is a small organic molecule, nucleic acid etc), the inhibitor is suitably covalently coupled directly or indirectly to the amino acid sequence of the second molecular component. Covalent coupling may be achieved by standard chemical methods depending on the chemical structure of the inhibitor utilized.

In one broad embodiment, the first molecular component comprises at least one protease cleavage site cleavable by the protease of the second molecular component, whereby cleavage by the protease releases inhibition of the enzyme activity of the first molecular component to thereby switch the first molecular component of the biosensor from a catalytically inactive to a catalytically active state. Suitably, the at least one protease cleavage site in the first molecular components is intermediate the enzyme amino acid sequence and the enzyme inhibitor.

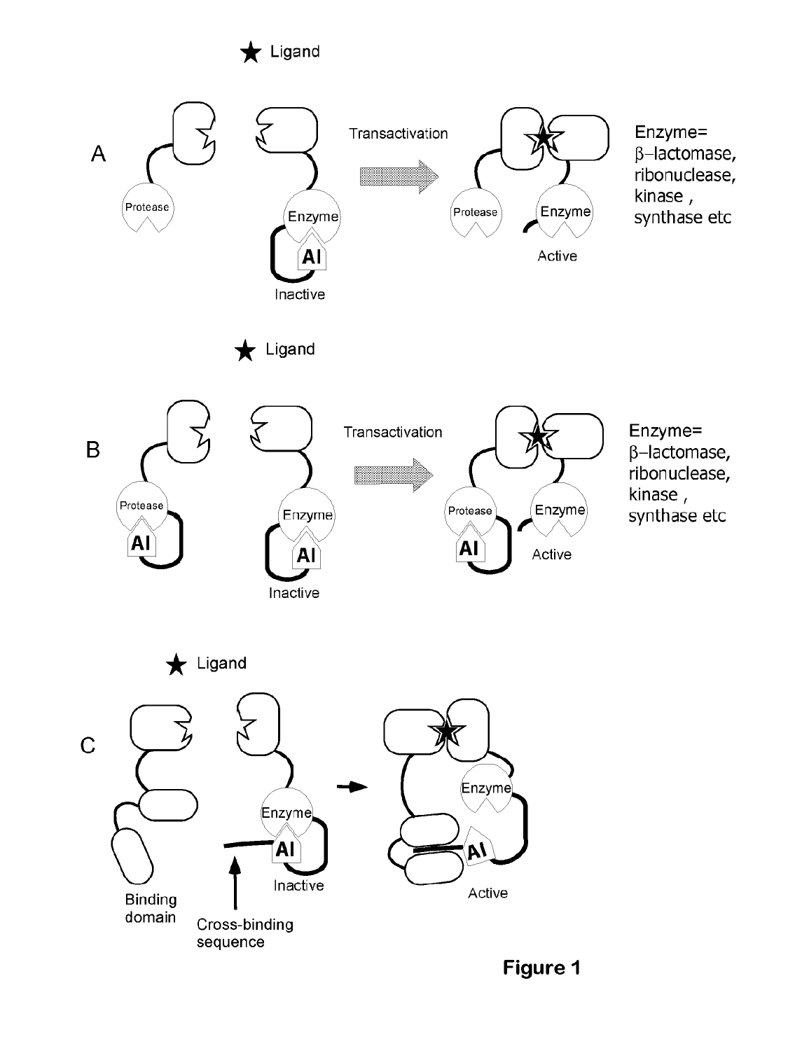

As previously described, the protease of the second molecular component may be constitutively active (see for example FIG. 1A) in the absence of a protease inhibitor. Co-localization of the first and second molecular components upon binding or interaction between the first binding partner, the second binding partner and in some cases a target molecule, spatially localizes this constitutive protease activity in the proximity of the first molecular component to enable cleavage of the protease cleavage site in the first molecular component, to thereby facilitate switching of the enzyme of the first molecular component from a catalytically inactive state to a catalytically active state.

In another particular embodiment, the second molecular component further comprise an inhibitor of said another protease, typically an autoinhibitor peptide as hereinbefore described. According to this embodiment (such as schematically shown in FIG. 1B), the protease of the second molecular component is partially inhibited and thereby possesses or displays a low or basal level of protease activity. Co-localization of the first and second molecular components upon binding or interaction between the first binding partner, the second binding partner and in some cases a target molecule, spatially localizes this low or basal protease activity in the proximity of the first molecular component to enable cleavage of the protease cleavage site in the first molecular component, thereby facilitate switching of the enzyme of the first molecular component from a catalytically inactive state to a catalytically active state.

In an alternative general embodiment, the second molecular component of the biosensor comprises an amino acid sequence other than a protease amino acid sequence. Preferably, the second molecular component of the biosensor comprises an amino acid sequence of a “trap” molecule and the first molecular component of the biosensor comprises an amino acid sequence of a “bait” molecule. Suitably, the bait molecule is connected, fused or linked to the amino acid sequence of the inhibitor of the non-protease enzyme. Typically, the bait molecule is located at or near the C-terminus of the first molecular component, although the bait molecule could be N-terminally located or located N-terminal and C-terminal of the enzyme inhibitor. Optionally, there is a linker amino acid sequence intermediate the bait amino acid sequence and the enzyme inhibitor. Binding of the bait molecule by the trap molecule occurs when the first and second molecular components are brought into proximity by a binding event between the respective first and second binding partners. Binding of the bait molecule by the trap molecule facilitates release of the inhibitor from the non-protease enzyme, thereby switching the non-protease enzyme from a catalytically active to a catalytically inactive state. A non-limiting example is schematically shown in FIG. 1C).

In some embodiments, the bait molecule may be or comprise amino acids (e.g. natural or non-natural amino acids) and peptides inclusive of chemically modified amino acids and peptides, peptides modified to include non-natural amino acids, PNA, single or double-stranded nucleic acids inclusive of DNA or RNA aptamers, carbohydrates, lipids, lectins and/or binding agents such as biotin or avidin, although without limitation thereto. As previously described, this bait amino acid sequence may be modified to include single- or double-stranded DNA, RNA, lipids, binding agents, chemical modifications to side chains etc.

Suitably, when the bait molecule is or comprises a peptide, it is typically of about 5-40 amino acids, preferably about 8 to about 30 amino acids or about 12-20 amino acids in length.

The bait molecule of the first molecular component and the trap molecule of the second molecular component may bind or interact by way of any molecular interaction. Non-limiting examples include: a protein:protein interaction where the bait molecule is a peptide and the trap molecule of the second molecular component comprises an amino acid sequence of a protein or fragment thereof; a nucleic acid: nucleic acid interaction where the bait molecule and the trap molecule comprise complementary nucleotide sequences; a biotin:avidin interaction wherein the bait molecule and the trap molecule respectively comprise avidin and biotin or vice versa; and a lectin: carbohydrate interaction wherein the bait molecule and the trap molecule respectively comprise a lectin and carbohydrate or vice versa, although without limitation thereto.

In another particular form of this embodiment, the trap molecule of the second molecular component comprises an amino acid sequence of an affinity clamp. The affinity clamp preferably comprises a recognition domain and, optionally, an enhancer domain. The recognition domain is typically capable of binding one or more target molecules, such as described in (i)-(xii) above. Recognition domains may include, but are not limited to, domains involved in phospho-tyrosine binding (e.g. SH2, PTB), phospho-serine binding (e.g. UIM, GAT, CUE, BTB/POZ, VHS, UBA, RING, HECT, WW, 14-3-3, Polo-box), phospho-threonine binding (e.g. FHA, WW, Polo-box), proline-rich region binding (e.g. EVH1, SH3, GYF), acetylated lysine binding (e.g. Bromo), methylated lysine binding (e.g. Chromo, PHD), apoptosis (e.g. BIR, TRAF, DED, Death, CARD, BH), cytoskeleton modulation (e.g. ADF, GEL, DH, CH, FH2), ubiquitin-binding domains or modified or engineered versions thereof, or other cellular functions (e.g. EH, CC, VHL, TUDOR, PUF Repeat, PAS, MH1, LRR1, IQ, HEAT, GRIP, TUBBY, SNARE, TPR, TIR, START, SOCS Box, SAM, RGS, PDZ, PB1, LIM, F-BOX, ENTH, EF-Hand, SHADOW, ARM, ANK).

The enhancer domain typically increases or enhances the binding affinity for at least one or the target molecules. In some embodiments, the affinity may be increased by at least 10, 100 or 1000 fold compared to that of the recognition domain alone. The affinity clamp may further comprise linker connecting the recognition domain and the enhancer domain.

In one particular embodiment, the affinity clamp comprises a recognition domain that comprises at least a portion or fragment of a PDZ domain and an enhancer domain that comprises at least a portion or fragment of a fibronectin type III domain. The PDZ domain may be derived from a human Erbin protein. Erbin-PDZ (ePDZ) binds to target molecules such as the C-termini of p120-related catenins (such as β-catenin and Armadillo repeat gene deleted in Velo-cardio-facial syndrome (ARVCF)). Preferably, this embodiment of the affinity claim further comprises the tenth (10^{th}) type III (FN3) domain of human fibronectin as an enhancer domain.

In some embodiments, the affinity clamp may comprise one or more connector amino acid sequences. For example, a connector amino acid sequence may connect the protease amino acid sequence (such as comprising a protease amino acid sequence) to the Erbin-PDZ domain, the Erbin-PDZ domain to the FN3 domain and/or the FN3 domain to the inhibitor.

Reference is also made to WO2009/062170, Zhuang & Liu, 2011, Comput. Theoret. Chem. 963 448, Huang et al, 2009, J. Mol. Biol. 392 1221 and Huang et al., 2008, PNAS (USA) 105 6578 for a more detailed explanation of affinity clamp structure and function.

According to this embodiment, the bait molecule of the first molecular component can be bound by, or interact with, the affinity clamp. In one particular embodimemt, the affinity clamp may be an ePDZ-FN3 affinity clamp. A non-limiting example of this embodiment is shown in FIG. 1C. The bait molecule may be or comprise a peptide corresponding to the C-terminal residues of ARVCF or β-catenin (NH₂-PQPVDSWV-COOH: SEQ ID NO:5; and NH₂-PASPDSWV-COOH: SEQ ID NO:6, respectively).

It will be appreciated that by addition of excess “free” bait molecule, it may displace the bait molecule of the first molecular component, thereby switching “off” the catalytic activity of the enzyme by allowing the inhibitor to re-bind the enzyme of the first molecular component.

Accordingly, in one preferred form the biosensor is a reversible biosensor.

As will be understood from the foregoing, a binding interaction between the first binding partner of the first molecular component and the second binding partner of the second molecular component suitably facilitates co-localization of the first and second molecular components. In one general embodiment, this facilitates at least partial release of inhibition of the non-protease enzyme of the first molecular component by the inhibitor to switch the enzyme of the first molecular component from a catalytically inactive state to a catalytically active state.

The first binding partner and/or the second binding partner may be or comprise binding moieties in the form of proteins, single- or double-stranded nucleic acids (e.g DNA or RNA), sugars, oligosaccharides, polysaccharides or other carbohydrates, lipids or any combinations of these such as glycoproteins, PNA constructs etc. By way of example only, binding moieties may be, or comprise: (i) an amino acid sequence of a ligand binding domain of a receptor responsive to binding of a target molecule such as a cognate growth factor, cytokine, a hormone (e.g. insulin), neurotransmitters etc; (ii) an amino acid sequence of an ion or metabolite transporter capable of, or responsive to, binding of a target molecule such as an ion or metabolite (e.g a Ca²⁺-binding protein such as calmodulin or calcineurin or a glucose transporter); (iii) a zinc finger amino acid sequence responsive to zinc-dependent binding a DNA target molecule; (iv) a helix-loop-helix amino acid sequence responsive to binding a DNA target molecule; (v) a pleckstrin homology domain amino acid sequence responsive to binding of a phosphoinositide target molecule; (vi) an amino acid sequence of a Src homology 2- or Src homology 3-domain responsive to a signaling protein; (vii) an amino acid sequence of an antigen responsive to binding of an antibody target molecule; or (viii) an amino acid sequence of a protein kinase or phosphatase responsive to binding of a phosphorylatable or phosphorylated target molecule; (ix) ubiquitin-binding domains; (x) proteins or protein domains that bind small molecules, drugs or antibiotics such as rapamycin-binding FKBP and FRB domains; (xi) single- or double-stranded DNA, RNA or PNA constructs that bind nucleic acid target molecules, such as where the DNA or RNA are coupled or cross-linked to an amino acid sequence or other protein-nucleic acid interaction; and/or (xii) an affinity clamp such as a PDZ-FH3 domain fusion; inclusive of modified or engineered versions thereof, although without limitation thereto.

It will also be appreciated that the first binding partner and/or the second binding partner may be modified or chemically derivatized such as with bindng agents such as biotin, avidin, epitope tags, lectins, carbohydrates, lipids although without limitation thereto.

It will further be appreciated that the molecules listed in (i)-(xii) could also be adapted for use as trap molecules and/or bait molecules as hereinbefore described.

Reference is also made to International Publication WO2015/035452 for non-limiting examples of molecules that may be suitable as binding partners, trap and bait molecules.

In some embodiments the first binding partner and the second binding partner may directly bind, interact or form a complex. The first binding partner and the second binding partner may comprise molecules that can directly bind or interact. Accordingly, the direct binding interaction between the target molecule and the first binding partner of the first molecular component and the second binding partner of the second molecular component suitably facilitates co-localization of the first and second molecular components.

In other embodiments, the first binding partner and the second binding partner are capable of binding, interacting or forming a complex with a target molecule. Typically, the first binding partner and the second binding partner are capable of binding, interacting or forming a complex with the same target molecule. By way of example, the first binding partner and the second binding partner may comprise amino acid sequences of respective proteins or protein domains or fragments that are capable of binding different portions or moieties of the same target molecule. In some embodiments, the first binding partner and the second binding partner are capable of co-operatively binding the target molecule. Accordingly, the binding interaction between the target molecule and the first binding partner of the first molecular component and the second binding partner of the second molecular component suitably facilitates co-localization of the first and second molecular components. It will also be appreciated that the “same” target molecule can have respective, different moieties, subunits, domains, ligands or epitopes that can be bound by the respective first and second binding partners to thereby co-localize and activate protease activity. Biosensors of this general type may be referred to as “dual specificity” biosensors.

In this regard, the target molecule may be any ligand, analyte, epitope, domain, fragment, subunit, moiety or combination thereof, such as a protein inclusive of antibodies and antibody fragments, antigens, phosphoproteins, glycoproteins, lipoproteins and glycoproteins, lipid, phospholipids, carbohydrates inclusive of simple sugars, disaccharides and polysaccharides, nucleic acids, nucleoprotein or any other molecule or analyte. These include drugs and other pharmaceuticals including antibiotics, chemotherapeutic agents and lead compounds in drug design and screening, molecules and analytes typically found in biological samples such as biomarkers, tumour and other antigens, receptors, DNA-binding proteins inclusive of transcription factors, hormones, neurotransmitters, growth factors, cytokines, receptors, metabolic enzymes, signaling molecules, nucleic acids such as DNA and RNA, membrane lipids and other cellular components, pathogen-derived molecules inclusive of viral, bacterial, protozoan, fungal and worm proteins, lipids, carbohydrates and nucleic acids, although without limitation thereto.

In one embodiment, the first and/or second binding partners comprise an amino acid sequence of at least a fragment of any protein or protein fragment or domain that can bind or interact directly, or bind to a target molecule. The binding partner may be, or comprise a protein such as a peptide, antibody, antibody fragment or any other protein scaffold that can be suitably engineered to create or comprise a binding portion, domain or region (e.g. reviewed in Binz et al., 2005 Nature Biotechnology, 23, 1257-68.) which binds a target molecule.

In one particular embodiment, the first binding partner and/or the second binding partner is or comprises an amino acid sequence of an affinity clamp, as hereinbefore described.

In another embodiment, the first binding partner and/or the second binding partner amino acid sequences comprise one or a plurality epitopes that can be bind or be bound by an antibody target molecule.

In another embodiment, the first binding partner and/or second binding partners may be or comprise an antibody or antibody fragment, inclusive of monoclonal and polyclonal antibodies, recombinant antibodies, Fab and Fab′2 fragments, diabodies and single chain antibody fragments (e.g. scVs), although without limitation thereto. Suitably, the first and second binding partners may be or comprise respective antibodies or antibody fragments that bind a target molecule.

In yet another particular embodiment, the first binding partner and/or second binding partner may be or comprise an antibody-binding molecule, wherein the antibody(ies) has specificity for a target molecule. The antibody-binding molecule is preferably an amino acid sequence of protein A, or a fragment thereof (e.g a ZZ domain), which binds an Fc portion of the antibody.

Non-limiting examples of binding partners are shown schematically in FIG. 1 and FIG. 2.

Suitably, the biosensor comprises a first molecular component that is a recombinant fusion protein and a second first molecular component that is a recombinant fusion protein. In some embodiments, the or each recombinant fusion protein comprises an affinity tag at a C-terminus thereof, which affinity tag facilitates isolation of biosensor molecules where protein translation has proceeded to the C-terminus of the protein product. The affinity tag suitably comprises an amino acid sequence of an epitope tag, fusion partner or other moiety that facilitates isolation and purification of the recombinant fusion protein.

Well known examples of fusion partners include, but are not limited to, glutathione-S-transferase (GST), maltose binding protein (MBP) and metal-binding moieties such as polyhistidine (e.g. HIS₆), for which affinity purification reagents are well known and readily available. Epitope tags are usually short peptide sequences for which a specific antibody is available. Well-known examples of epitope tags for which specific monoclonal antibodies are readily available include c-myc, influenza virus haemagglutinin and FLAG tags.

Preferably, the affinity tag is a C-terminal hexahistidine (HIS₆) tag.

Particular embodiments of the biosensor comprise first and/or second molecular components, non-protease enzymes and inhibitors thereof, trap molecules, bait molecules proteases and/or protease inhibitors that comprise an amino acid sequence set forth in any one of SEQ ID NOS:1 and 2.

It will also be appreciated that the invention includes biosensors that comprise first and/or second molecular components, cross-binders, subcomponents, proteases and/or protease inhibitors that comprise amino acid sequences that are variants of the amino acid sequences set forth in SEQ ID NOS:1 and 2 and/or fragments thereof. Typically, such variants have at least 80%, at least 85%, at least 90%, 91%, 92%, 93%, 94% 95%96%, 97%, 98% or 99% sequence identity with any of the amino acid sequences set forth in SEQ ID NOS:1 and 2. By way of example only, conservative amino acid variations may be made without an appreciable or substantial change in function. For example, conservative amino acid substitutions may be tolerated where charge, hydrophilicity, hydrophobicity, side chain “bulk”, secondary and/or tertiary structure (e.g. helicity), target molecule binding, protease activity and/or protease inhibitory activity are substantially unaltered or are altered to a degree that does not appreciably or substantially compromise the function of the biosensor and/or the first or second molecular components.

The term “sequence identity” is used herein in its broadest sense to include the number of exact amino acid matches having regard to an appropriate alignment using a standard algorithm, having regard to the extent that sequences are identical over a window of comparison. Sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999). Suitably, sequence identity is measured over the entire length of any one of SEQ ID NOS:1 and 2.

Protein fragments may comprise up to 5%, 10%, 15%, 20%, 25%, 30%, 35%, 40%, 45%, 50%, 55%, 60%, 65%, 70%, 75%, 80%, 85%, 90% or up to 95-99% of an amino acid sequence set forth in any one of SEQ ID NOS:1 and 2. In some embodiments, the protein fragment may comprise up to 5, 10, 15, 20, 30, 40, 50, 60, 70, 80, 90, 100, 120, 150, 180 200, 220, 230, 250, 280, 300, 320, 350, 400 or more amino acids of an amino acid sequence set forth in any one of SEQ ID NOS:1 and 2.

It will be appreciated from the foregoing that the biosensor disclosed herein is comprises a non-protease enzyme that is capable of switching from a catalytically inactive state to a catalytically active state in response to a binding interaction or event that occurs between the first and second binding partners, such as when binding a target molecule. Suitably, the catalytically active enzyme is capable of reacting with a substrate molecule to produce a detectable signal. The detectable signal may be or include chromogenic, fluorescent, light (e.g. bioluminescent), electrical, radioactive and other detectable signals.

Examples of chromogenic substrate molecules include diaminobenzidine (DAB), permanent red, 3-ethylbenzthiazoline sulfonic acid (ABTS), 5-bromo-4-chloro-3-indolyl phosphate (BCIP), nitro blue tetrazolium (NBT), 3,3′,5,5′-tetramethyl benzidine (TNB) and 4-chloro-1-naphthol (4-CN) and nitrocefin, although without limitation thereto. As will be described in more detail in the Examples, β-lactamase cleavage of nitrocefin results in an increase in detectable signal measured as an increase in absorbance at A₆₂₀.

In embodiments where the detectable signal is electrical (i.e the production of electrons), preferably the enzyme of the first molecular component is an oxidoreductase such as GDH, LDH or DHFR. According to these embodiments, the substrate molecule is respectively glucose, lactate or dihydrofolic acid.

A non-limiting example of a chemiluminescent substrate molecule is Luminol™, which is oxidized in the presence of horseradish peroxidase and hydrogen peroxide to form an excited state product (3-aminophthalate).

Fluorescent signals may be produced by fluorophores such as a coumarin, fluorescein isothiocyanate (FITC), tetramethylrhodamine isothiocyanate (TRITC), allophycocyanin (APC), Texas Red (TR), TAMRA, LC red, HEX, FAM, TET, ROX, Cy dyes such as Cy3 or Cy5 or R-Phycoerythrin (RPE) or derivatives thereof, although without limitation thereto.

In a preferred embodiment where the substrate molecule comprises a fluorophore, the substrate molecule may be quenched, whereby release of the fluorophore from quenching is detected as an increase in fluorescence signal. Non-limiting examples of quenchers include 5-amino-2-nitrobenzoic acid (ANA), Deep Dark Quenchers (DDQ), Iowa Black quenchers, Black Hole quenchers, Eclipse quenchers, Dabcyl and QSY quenchers which are commercially available from sources such as Eurogentec, Integrated DNA Technologies and Molecular Probes.

Measurement and/or detection of signals resulting from a binding event between the first and second molecular component may be performed or achieved by any method known in the art. A non-limiting example is provided in the Examples.

A further aspect of the invention provides a kit or composition comprising one or more biosensors disclosed herein in combination with one or more substrates.

The biosensor disclosed herein is particularly suitable for detection of a target molecule. The target molecule may be any molecule which can be detected by the first binding partner and the second binding partner of the biosensor, such as hereinbefore described.

It will be appreciated that in certain aspects, the biosensor disclosed herein may have efficacy in molecular diagnostics wherein the “target molecule” is an analyte or other molecule of diagnostic value or importance.

In a further aspect, the invention provides a method of detecting a target molecule, said method including the step of contacting the composition of the aforementioned aspect with a sample to thereby determine the presence or absence of the target molecule in the sample.

Suitably, the sample is a biological sample. Biological samples may include organ samples, tissue samples, cellular samples, fluid samples or any other sample obtainable, obtained, derivable or derived from an organism or a component of the organism. The biological sample can comprise a fermentation medium, feedstock or food product such as for example, but not limited to, dairy products.

In particular embodiments, the biological sample is obtainable from a mammal, preferably a human. By way of example, the biological sample may be a fluid sample such as blood, serum, plasma, urine, saliva, cerebrospinal fluid or amniotic fluid, a tissue sample such as a tissue or organ biopsy or may be a cellular sample such as a sample comprising red blood cells, lymphocytes, tumour cells or skin cells, although without limitation thereto. A particular type of biological sample is a pathology sample.

Suitably, the activity of the biosensor is not substantially inhibited by components of the sample (e.g. serum proteins, metabolites, cells, cellular debris and components, naturally-occurring protease inhibitors etc). Embodiments where the protease is of Potyvirus origin such as hereinbefore described may be particularly resistant to inhibition by components of human or mammalian biological samples.

In one embodiment, the biosensor and/or methods of use may be applicable to drug testing such as for detecting the use of illicit drugs of addiction (e.g cannabinoids, amphetamines, cocaine, heroin etc.) and/or for the detection of performance-enhancing substances in sport and/or masking agents that are typically used to avoid detection of performance-enhancing substances. This may be applicable to the detection of banned performance-enhancing substances in humans and/or other mammals such as racehorses and greyhounds that may be subjected to illicit “doping” to enhance performance.

In another embodiment, the biosensor and/or methods of use may be for diagnosis of a disease or condition of an organism, inclusive of plants and animals. Animals may include fish, avians (e.g poultry) and mammals such as humans, livestock (e.g cattle and sheep), domestic pets (e.g. cats and dogs), performance animals (e.g. racehorses) and laboratory animals (e.g. rats, mice and rabbits), although without limitation thereto.

A preferred aspect of the invention provides a method of diagnosis of a disease or condition in a mammal, such as a human, said method including the step of contacting the composition of the aforementioned aspect with a biological sample obtained from the mammal or human to thereby determine the presence or absence of a target molecule in the biological sample, determination of the presence or absence of the target molecule facilitating diagnosis of the disease or condition.

The disease or condition may be any, where detection of a target molecule assists diagnosis. Non limiting examples of target molecules or analytes include blood coagulation factors such as previously described, kallikreins inclusive of PSA, matrix metalloproteinases, viral and bacterial proteases, antibodies, glucose, triglycerides, lipoproteins, cholesterol, tumour antigens, lymphocyte antigens, autoantigens and autoantibodies, drugs, drug precursors and drug metabolites, salts, creatinine, blood serum or plasma proteins, pesticides, uric acid, products and intermediates of human and animal metabolism and metals.

This preferred aspect of the invention may be adapted to be performed as a “point of care” method whereby determination of the presence or absence of the target molecule may occur at a patient location which is then either analysed at that location or transmitted to a remote location for diagnosis of the disease or condition.

One particular aspect of the invention therefore provides a device comprising the biosensor disclosed herein in a chamber or cell of the device and, optionally, an amplifier molecule. In some embodiments, the cell or chamber may be a component of, or connected or coupled to, a “point of care” device such as hereinbefore described.

Suitably, the cell or chamber is perfused with a sample and protease activity is detected.

In one form, the device may be for providing a disease diagnosis from a diagnostic test result, the device comprising:

a processor; and

a memory coupled to the processor, the memory including computer readable program code components that, when executed by the processor, perform a set of functions including:

analysing a diagnostic test result and providing a diagnosis of the disease or condition.

The device may also be suitable for communicating a diagnostic test result, the device comprising:

a processor; and

a memory coupled to the processor, the memory including computer readable program code components that, when executed by the processor, perform a set of functions including:

transmitting a diagnostic target result to a receiving device; and

optionally receiving a diagnosis of the disease or condition from the or another receiving device.

The device may be in the form of a mobile or cellular phone, a computer or any other electronic device capable of analysing diagnostic target results at the “point of care” or transmitting and/or receiving information (i.e. diagnostic target results and a disease diagnosis) to or from a receiving device at a remote location.

In one embodiment, protease activity is detected electrochemically. For example, detection may be by digesting a protein or peptide clot covering the surface of the electrode, whereby protease digestion of the clot enables access of an electrolyte to the electrode. In another example, activating the enzyme changes conductivity of a solution in the cell. In yet another example, the protease activity of the biosensor digests a conducting substrate and thereby changes conductivity. In a further example, the protease activity of the biosensor induces a substrate molecule or enzyme to become electrochemically active.

In another embodiment, protease activity is detected acoustically. For example, detection may be by measuring propagation of sound waves due to changes in viscosity of gels and solutions comprising one or more substrates of the protease.

In another embodiment, protease activity is detected optically. For example, detection may be by monitoring changes in reflection or refraction of light from surfaces comprising (e.g coated or impregnated with) one or more substrates of the protease.

A further embodiment of the invention relates to imaging of biological molecules. The activated protease activity of the biosensor cleaves a substrate peptide designed to change fluorescence and circulation time upon cleavage. This, for instance, may be brought about by the exposure of hydrophobic, or a cell-penetrating sequence and dequenching of a fluorophore. Alternatively the substrate peptide may be modified with a contrast substance such as metal (Ba) or an isotope for whole body imaging.

An advantage of the invention over the targeting of a particular tumour protease directly is in signal amplification and standardisation of the targeting peptide. Further the specificity of the response may be increased by targeting of the biosensor to a particular cell type or surface antigen by fusing or conjugating it to a targeting domain comprising a peptide, antibody or other targeting molecule.

In a further embodiment, the biosensor comprises first and second binding partners targeted to a particular type of surface molecule such as, for example, EGF receptor enriched in certain tumours. Activation of the proteolytic activity of the biosensor can be used for tumour visualisation or therapeutic targeting.

In a still further embodiment, an array of biosensors is connected or coupled to one or more electronic devices that utilise the ‘point of care” diagnostic device for identification of infective species. This embodiment is based on the observation that surface and secreted proteases play a key role in invasion and propagation of metazoan, bacterial and viral parasites. Each infective species can be categorized according to the unique protease signature. In a variation of the above described embodiment, the sensor array is composed of biosensors activated by metabolites and/or proteins of a parasitic organism.

Diagnostic aspects of the invention may also be in the form of a kit comprising one or a plurality of different biosensors capable of detecting one or a plurality of different target molecules. In this regard, a kit may comprise an array of different biosensors capable of detecting a plurality of different target molecules. The kit may further comprise one or more amplifier molecules, deactivating molecules and/or labeled substrates, as hereinbefore described. The kit may also comprise additional components including reagents such as buffers and diluents, reaction vessels and instructions for use.

A further aspect of the invention provides an isolated nucleic acid which encodes an amino acid sequence of the biosensor of the invention, respective first and second molecular components or a variant thereof as hereinbefore defined.

The term “nucleic acid” as used herein designates single-or double-stranded mRNA, RNA, cRNA, RNAi, siRNA and DNA inclusive of cDNA, mitochondrial DNA (mtDNA) and genomic DNA.

A “polynucleotide” is a nucleic acid having eighty (80) or more contiguous nucleotides, while an “oligonucleotide” has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

In particular embodiments, the isolated nucleic acid encodes an amino acid sequence selected from the group consisting of: SEQ ID NOS:1 and 2, or a fragment or variant thereof.

The invention also provides “genetic constructs” that comprise one or more isolated nucleic acids, variants or fragments thereof as disclosed herein operably linked to one or more additional nucleotide sequences.

As generally used herein, a “genetic construct” is an artificially created nucleic acid that incorporates, and/or facilitates use of, an isolated nucleic acid disclosed herein.

In particular embodiments, such constructs may be useful for recombinant manipulation, propagation, amplification, homologous recombination and/or expression of said isolated nucleic acid.

As used herein, a genetic construct used for recombinant protein expression is referred to as an “expression construct”, wherein the isolated nucleic acid to be expressed is operably linked or operably connected to one or more additional nucleotide sequences in an expression vector.

An “expression vector” may be either a self-replicating extra-chromosomal vector such as a plasmid, or a vector that integrates into a host genome.

In this context, the one or more additional nucleotide sequences are regulatory nucleotide sequences.

By “operably linked” or “operably connected” is meant that said regulatory nucleotide sequence(s) is/are positioned relative to the nucleic acid to be expressed to initiate, regulate or otherwise control expression of the nucleic acid.

Regulatory nucleotide sequences will generally be appropriate for the host cell used for expression. Numerous types of appropriate expression vectors and suitable regulatory sequences are known in the art for a variety of host cells.

One or more regulatory nucleotide sequences may include, but are not limited to, promoter sequences, leader or signal sequences, ribosomal binding sites, transcriptional start and termination sequences, translational start and termination sequences, splice donor/acceptor sequences and enhancer or activator sequences.

Constitutive or inducible promoters as known in the art may be used and include, for example, nisin-inducible, tetracycline-repressible, IPTG-inducible, alcohol-inducible, acid-inducible and/or metal-inducible promoters.

In one embodiment, the expression vector comprises a selectable marker gene. Selectable markers are useful whether for the purposes of selection of transformed bacteria (such as bla, kanR, ermB and tetR) or transformed mammalian cells (such as hygromycin, G418 and puromycin resistance).

Suitable host cells for expression may be prokaryotic or eukaryotic, such as bacterial cells inclusive of Escherichia coli (DH5α for example), yeast cells such as S. cerivisiae or Pichia pastoris, insect cells such as SF9 cells utilized with a baculovirus expression system, or any of various mammalian or other animal host cells such as CHO, BHK or 293 cells, although without limitation thereto.

Introduction of expression constructs into suitable host cells may be by way of techniques including but not limited to electroporation, heat shock, calcium phosphate precipitation, DEAE dextran-mediated transfection, liposome-based transfection (e.g. lipofectin, lipofectamine), protoplast fusion, microinjection or microparticle bombardment, as are well known in the art.

Purification of the recombinant biosensor molecule may be performed by any method known in the art. In preferred embodiments, the recombinant biosensor molecule comprises a fusion partner (preferably a C-terminal His tag) which allows purification by virtue of an appropriate affinity matrix, which in the case of a His tag would be a nickel matrix or resin.

While many of the aforementioned aspects and embodiments relate to molecular diagnostics, it will also be appreciated that in certain aspects, the biosensor disclosed herein may have efficacy in cellular engineering where it is employed as extracellular, membrane, intracellular or nuclear receptor detecting a natural or synthetic ligand. Activation of the protease may be actuated on an effector comprising an enzymatic or structural protein domain operably linked to an auto inhibitory domain via a linker containing a cleavage site of the said protease. Such a protein domain may be a variant of a natural or synthetic protease, kinase, phosphatase, aminase, nuclease, scaffolding protein, structural protein, transcription factor or RNA binding protein, although without limitation thereto. Activation of the said effect may regulate a natural or synthetic enzymatic, metabolic or signalling cascade modulating cellular processes such as cellular proliferation, migration, biosynthesis survival, differentiation or death, although without limitation thereto

So that the invention may be readily understood and put into practical effect, embodiments of the invention will be described with reference to the following non-limiting Examples.

### EXAMPLES

A two-component biosensor schematically depicted in FIG. 2 was produced by bacterially expressing respective, recombinant fusion proteins comprising the following amino acid sequences.

### First Molecular Component:

BLA^{TVMV}-BLIP-FRB-His
(SEQ ID NO: 1)
MGGSGGHPETLVKVKDAEDQLGARVGYIELDLNSGKILESFRPEERFPMMSTF
KVLLCGAVLSRIDAGQEQLGRRIHYSQNDLVEYSPVTEKHLTDGMTVRELCSA
AITMSDNTAANLLLTTIGGPKELTAFLHNMGDHVTRLDRWEPELNEAIPNDERD
TTMPVAMATTLRKLLTGELLTLASRQQLIDWMEAKVAGPLLRSALPAGWFIAD
KSGAGERGSRGIIAALGPDGKPSRIVVIYTTGSQATMDERNRQIAEIGASLIKHW
GGSGETVRFQSGSEKIRLRGGAGVMTGAKFTQIQFGMTRQQVLDIAGAENCET
GGSFGDSIHCRGHAAGAYYAYATFGFTSAAADAKVDSKSQEKLLAPSAPTLTL
AKFNQVTVGMTRAQVLATVGQGSCTTWSEYYPAYPSTAGVTLSLSCFDVDGY

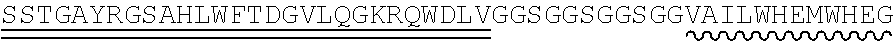

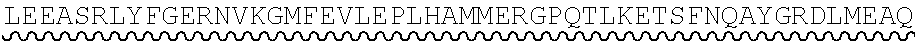

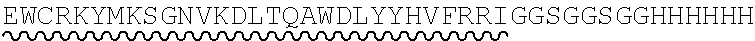

Second molecular component:
FKBP12-TVMV^{Thr}-AI-His
(SEQ ID NO: 2)

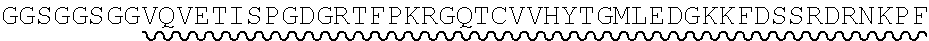

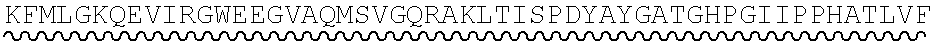

DVELLKLEGGSGGSGGSGGSGGSGGSKALLKGVRDFNPISACVCLLENSSDGHS
ERLFGIGFGPYIIANQHLFRRNNGELTIKTMHGEFKVKNSTQLQMKPVEGRDIIV
IKMAKDFPPFPQKLKFRQPTIKDRVCMVSTNFQQKSVSSLVSESSHIVHKEDTSF
WQHWITTKDGQCGSPLVSIIDGNILGIHSLTHTTNGSNYFVEFPEKFVATYLDAA
DGWCKNWKFNADKISWGSFILWEDAPEDFMSGLVPRGVGREYVRFAPGSTHH
HHHH

The TVMV protease and β-lactamase amino acid sequences are underlined, the amino acid sequence of the protease cleavage site is bolded and the amino acid sequence of the autoinhibitor peptide is double-underlined. The rapamycin binding domains FRB and FKBP12 are wiggled. The His tag is at the C-terminus. Linker sequences are in plain font.

The recombinant fusion proteins were produced in E. coli bacteria and purified using a nickel resin affinity chromatography according to previously published procedures (e.g. for cytoplasmic expression and purification of TVMV-based constructs see Stein et al., PNAS, 2014, 111, 15934-9; for periplasmic expression and purification of the BLA-based construct see Nirantar et al. Biosensors and Bioelectronics, 2013, 47, 421-8).

β lactamase activity in response to rapamycin as a target molecule was measured by the following assay:

### 200 μL Total Assay Volume:

The reaction buffer contained phosphate buffered saline (PBS) (137 mM NaCl, 2.7 mM KCl, 10 mM Na₂HPO₄ and 2 mM KH₂PO₄, pH 7.4) supplemented with 50 μg/mL bovine serum albumin (BSA)
β lactamase activity was assayed with 25 μM Nitrocefin monitoring its increase in absorbance at A₆₂₀
BLA^{TVMV}-BLIP-FRB, FKBP12-TVMV^{Thr}-AI and FKBP12-TVMV (which was obtained by pre-treating FKBP12-TVMV^{Thr}-AI with 1U thrombin for 1 hour) were applied at the concentrations indicated
Rapamycin was included at the concentrations indicated
The reaction was initiated upon the addition of a solution of BLA^{TVMV}-BLIP-FRB pre-mixed with varying concentrations of rapamycin to a solution containing either FKBP12-TVMV^{Thr}-AI or FKBP12-TVMV pre-mixed with nitrocefin.

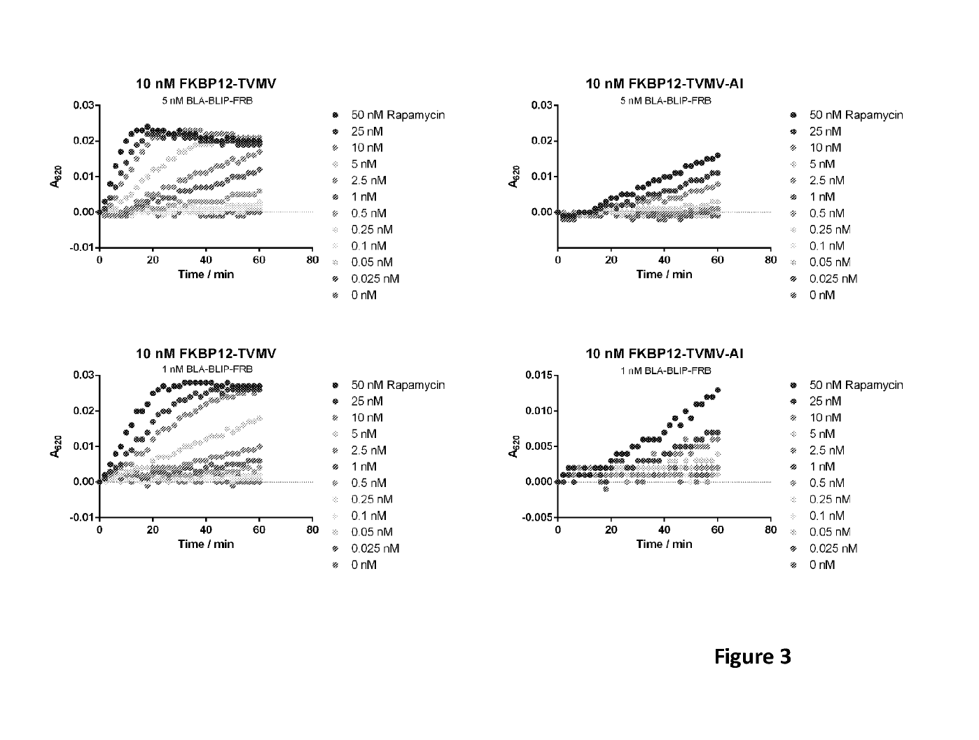

As shown in FIG. 3, dose-dependent detection of rapamycin was achieved by the biosensor, as measured by the hydrolysis of nitrocefin.

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

1.-36. (canceled)

37. A composition comprising:
(i) a biosensor comprising a first molecular component and a second molecular component, wherein
the first molecular component comprises: a first binding partner, an amino acid sequence of an enzyme that is not a protease; and an inhibitor of the enzyme; and
the second molecular component comprises: a second binding partner and an amino acid sequence capable of facilitating at least partial release of inhibition of the enzyme of the first molecular component by the inhibitor to thereby switch the enzyme of the first molecular component from a catalytically inactive state to a catalytically active state;
(ii) the biosensor of (i) and a substrate molecule;
(iii) an isolated nucleic acid encoding the first molecular component or the second molecular component of the biosensor of (i);
(iv) a genetic construct comprising the isolated nucleic acid of (iii); or
(v) a host cell comprising the genetic construct of (iv).

38. The composition of claim 37(i), whereby in a catalytically active state, the enzyme that is not a protease is capable of reacting with, or acting upon, a substrate molecule to produce detectable signal; optionally wherein the detectable signal is chromogenic, electrical, fluorescent or bioluminescent.

39. The composition of claim 37(i), wherein the enzyme that is not a protease is selected from the group consisting of: β-lactamase, β-galactosidase, glucose oxidase, glucose dehydrogenase, flavin adenine dinucleotide-dependent glucose dehydrogenase (FADGDH) and pyranose dehydrongease (PDH), lysozyme, malate dehydrogenase, horseradish peroxidase, alkaline phosphatase, luciferase, a transferase, an ATPase, a nuclease, a kinase, a synthase and an oxidoreductase.

40. The composition of claim 37(i), wherein the enzyme that is not a protease is β-lactamase; optionally wherein the inhibitor is a β-lactamase inhibitory peptide (BLIP).

41. The composition of claim 37(i), wherein the first binding partner and the second binding partner are capable of directly binding, coupling, interacting or forming a complex to thereby co-localize the first molecular component and the second molecular component; optionally wherein the first binding partner and the second binding partner are capable of co-operatively binding the target molecule.

42. The composition of claim 37(i), wherein the first binding partner and the second binding partner are capable of binding, coupling, interacting or forming a complex with a target molecule to thereby co-localize the first molecular component and the second molecular component; optionally wherein the first binding partner and the second binding partner are capable of co-operatively binding the target molecule.

43. The composition of claim 37(i), wherein the first binding partner and/or the second binding partner is or comprises an antibody or antibody fragment.

44. The composition of claim 37(i), wherein the first binding partner and/or the second binding partner is or comprises one or a plurality of epitopes.

45. The composition of claim 37(i), wherein the amino acid sequence capable of facilitating at least partial release of inhibition of the enzyme of the first molecular component is, or comprises, an amino acid sequence of a protease or fragment thereof; optionally wherein:
(a) the protease or fragment thereof is constitutively active;
(b) the protease or fragment thereof is at least partially inhibited; and/or
(c) the second molecular component further comprises an inhibitor of the protease or fragment thereof.

46. The composition of claim 37(i), wherein the amino acid sequence capable of facilitating at least partial release of inhibition of the enzyme of the first molecular component is, or comprises, an amino acid sequence of a protease or fragment thereof and wherein the first molecular component further comprises at least one protease cleavage site cleavable by said protease or fragment thereof of the second molecular component to at least partly release inhibition of the enzyme of the first molecular component by the inhibitor and thereby switch the enzyme from a catalytically inactive to a catalytically active state; optionally wherein:
(a) the protease or fragment thereof is constitutively active;
(b) the protease or fragment thereof is at least partially inhibited; and/or
(c) the second molecular component further comprises an inhibitor of the protease or fragment thereof.

47. The composition of claim 37(i), wherein the amino acid sequence capable of facilitating at least partial release of inhibition of the enzyme of the first molecular component is, or comprises, an amino acid sequence of a protease or fragment thereof; optionally wherein the protease or fragment thereof is of a virus; optionally wherein:
(a) the virus is a Potyvirus or a Flavivirus;
(b) the virus is a Potyvirus and is SMV, TEV or TVMV;
(c) the virus is a Flavivirus and is HCV;
(d) the protease is an NIa protease; or
(e) the protease is a SUMO-specific protease.

48. The composition of claim 37(i), wherein the amino acid sequence capable of facilitating at least partial release of inhibition of the enzyme of the first molecular component is, or comprises, an amino acid sequence of a protease or fragment thereof; optionally wherein the protease or fragment thereof is of a virus and wherein the first molecular component further comprises at least one protease cleavage site cleavable by said protease or fragment thereof of the second molecular component to at least partly release inhibition of the enzyme of the first molecular component by the inhibitor and thereby switch the enzyme from a catalytically inactive to a catalytically active state; optionally wherein:
(a) the virus is a Potyvirus or a Flavivirus;
(b) the virus is a Potyvirus and is SMV, TEV or TVMV;
(c) the virus is a Flavivirus and is HCV;
(d) the protease is an NIa protease; or
(e) the protease is a SUMO-specific protease.

49. The composition of claim 37(i), wherein the second molecular component does not comprise an amino acid sequence of a protease or fragment thereof.

50. The composition of claim 37(i), wherein the second molecular component does not comprise an amino acid sequence of a protease or fragment thereof and wherein the second molecular component comprises a trap molecule;
optionally wherein the trap molecule is an affinity clamp.

51. The composition of claim 37(i), wherein the second molecular component does not comprise an amino acid sequence of a protease or fragment thereof and wherein the second molecular component comprises a trap molecule and wherein the first molecular component comprises a bait molecule that is capable of binding or interacting with the trap molecule of the second molecular component;
optionally_wherein the trap molecule is an affinity clamp; optionally wherein the bait molecule is a peptide capable of binding or interacting with the affinity clamp.

52. The composition of claim 37(i), wherein the second molecular component does not comprise an amino acid sequence of a protease or fragment thereof and wherein the second molecular component comprises a trap molecule and wherein the first molecular component comprises a bait molecule that is capable of binding or interacting with the trap molecule of the second molecular component and wherein upon a binding interaction between the first binding partner, the second binding partner and optionally, a target molecule, the trap molecule is capable of binding the bait molecule to thereby at least partly release inhibition of the enzyme of the first molecular component by the inhibitor to thereby switch the enzyme of the first molecular component from a catalytically inactive state to a catalytically active state; optionally wherein the trap molecule is an affinity clamp; optionally wherein the bait molecule is a peptide capable of binding or interacting with the affinity clamp.

53. A method of detecting a target molecule, said method including the step of contacting the composition of claim 37(i) with a sample to thereby determine the presence or absence of a target molecule in the sample.

54. A method of diagnosing a disease or condition in an organism, said method including the step of contacting the composition of claim 37(i) with a biological sample obtained from the organism to thereby determine the presence or absence of a target molecule in the biological sample, and diagnosing the disease or condition by determination of the presence or absence of the target molecule.

55. The method of claim 54, wherein the organism is an animal; optionally wherein the animal is a human or other mammal.


SEQUENCE LISTING (6 sequences)

SEQ ID NO: 1 - protein, 572 aa, Artificial Sequence
  First molecular component, BLA(TVMV)-BLIP-FRB-His
MGGSGGHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEAKVAGPLLRSALPAGWFIADKSGAGERGSRGIIAALGPDGKPSRIVVIYTTGSQATMDERNRQIAEIGASLIKHWGGSGETVRFQSGSEKIRLRGGAGVMTGAKFTQIQFGMTRQQVLDIAGAENCETGGSFGDSIHCRGHAAGAYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGGSGGSGGSGGVAILWHEMWHEGLEEASRLYFGERNVKGMFEVLEPLHAMMERGPQTLKETSFNQAYGRDLMEAQEWCRKYMKSGNVKDLTQAWDLYYHVFRRIGGSGGSGGHHHHHH

SEQ ID NO: 2 - protein, 381 aa, Artificial Sequence
  Second molecular component, FKBP12-TVMV(Thr)-AI-His
GGSGGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGSGGSGGSGGSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMSGLVPRGVGREYVRFAPGSTHHHHHH

SEQ ID NO: 3 - protein, 10 aa, Artificial Sequence
  Segment of site F autoinhibitory peptide
RETVRFQSDT

SEQ ID NO: 4 - protein, 10 aa, Artificial Sequence
  peptide-based active site binder
DELILCPLDL

SEQ ID NO: 5 - protein, 8 aa, Artificial Sequence
  a peptide corresponding to the C-terminal residues of ARVCF
PQPVDSWV

SEQ ID NO: 6 - protein, 8 aa, Artificial Sequence
  a peptide corresponding to the C-terminal residues of delta-catenin
PASPDSWV